(19) United States
(12) Patent Application Publication
(10) Pub. No.: US 2009/0176690 A1
(43) Pub. Date: Jul. 9, 2009

(54) Composition and Method of Increasing the Substantivity of a Fragrance

(75) Inventors: Christophe Laudamiel, New York, NY (US); Joel Regis Claude Palix, Paris (FR); Christoph Hornetz, Berlin (DE); Nicole Dalmasso, Courbevoie (FR)

Correspondence Address: INTERNATIONAL FLAVORS & FRAGRANCES INC., 521 WEST 57TH ST, NEW YORK, NY, 10019, US

(73) Assignee: International Flavors & Fragrances Inc., New York, NY (US)

(21) Appl. No.: 12/333,456

(22) Filed: Dec. 12, 2008

Related U.S. Application Data
(60) Provisional application No. 61/013,675, filed on Dec. 14, 2007.

(51) Int. Cl. A61K 8/58; A61Q 13/00

(52) U.S. Cl. 512/2

(57) ABSTRACT

This invention relates to a composition and method of increasing the substantivity of a fragrance deposited on to a human substrate and to a fluid composition for use therein.

### STATUS OF RELATED APPLICATIONS

This application is a non-provisional application claiming priority of provisional U.S. Ser. No. 61/013,675, filed on Dec. 14, 2007, the contents hereby incorporated by reference as if set forth in its entirety.

### FIELD OF THE INVENTION

This invention relates to a composition and method of increasing the substantivity of a fragrance deposited on to a human substrate and to a fluid composition for use therein.

### BACKGROUND OF THE INVENTION

There is an ongoing need in the fragrance industry to increase the substantivity of fragrance composition to increase the longevity of the fragrances for consumers.

It is known in the art is that a fragrance is built according to the following pattern, a top note, also known as a head note, a middle note also called heart note and a base note, also called dry-down note or sould note as described for instance in “Perfume Legends”, 1996, by Michael Edwards or explained in “All in a Day's Work, Career using Science”, 2007, pp 73-74, by Megan Sullivan and edited by the National Science Teachers' Association.

The biggest challenge of perfumery is the lack of the prediction. The explanations are always found after the facts. Traditionally, scents are classified as notes based on their olfactory character. Top notes are detected and fade first, providing freshness, such as light scents that are usually citrus or wet greens lasting five to thirty minutes. Middle notes last sometimes a few hours and are the most prominent within the fragrances, usually combinations of floral, spicy, or fruit scents. Base notes give a perfume depth, last the longest, and are generally musky or woodsy notes. The classification of top, middle, and base notes designed to give a particular harmony is presently being challenged with a more exact approach.

It has been previously disclosed in the art to increase substantivity by including silicones into the base of a perfume product. United States No. 5,623,017 to Hill, assigned to Dow Corning Corporation, discloses a clear silicone gel which is used to improve the characteristics of various cosmetic products. One such cosmetic product is disclosed in U.S. Pat. No. 5,919,437 to Lee, et al., assigned to Colgate-Palmolive Company, which utilizes a combination of the silicone gel material with an active ingredient such as deodorant, antiperspirant, sunscreen, insect repellant or anti-fungal agent. Also see U.S. Pat. No. 4,673,570 to Soldati, assigned to Carter-Wallace, Inc., for an antiperspirant gel composition.

U.S. Pat. No. 5,654,362 to Schulz, Jr., et al., assigned to Dow-Corning Corporation, discloses a silicone oil and solvent composition for use in cosmetics.

United States Nos. 5,623,017 and 5,654,362 are incorporated here by reference for their teaching of cyclomethicone/dimethicone crosspolymer gels.

Nowhere in the prior art has it been disclosed that one may combine a particular fragrance with a range of other fragrances to increase substantivity without the two fragrances interfering and clashing with each other. Accordingly, it has been surprisingly discovered that a fragrance can be created to be combined with a large array of existing fragrances for an enhanced effect and increased substantivity.

### SUMMARY OF THE INVENTION

According to one embodiment of the invention, a fragrance primer is disclosed comprising fragrance material and a suitable carrier wherein the fragrance material comprises up to 50% of a fruity material, up to 90% of a floral material, up to 50% of a marine/aldehydic/ozonic material, up to 50% of an animalic leathery material, up to 90% of a musky material, up to 90% of a woody material, up to 90% of a sweet ambery material, up to 90% of a citrus material, up to 50% of a green material, up to 50% of a spicy material and up to 50% of a herbaceous material.

In yet another embodiment of the invention, a method of increasing the substantivity of a fragrance is disclosed comprising the steps of applying a fragrance primer to the skin with an applicator comprising a fragrance formulation in a suitable carrier and then applying a fragrance formulation over the fragrance primer.

### BRIEF DESCRIPTION OF THE DRAWINGS

FIG. I illustrates the substantivity of the fragrance primer by itself.

FIG. I illustrates the increased substantivity of a commercially available soft, floral, rich, and bouquet fragrance when used in combination with the fragrance primer.

FIG. III illustrates the increased substantivity of a commercially available soft floral classical bouquet fragrance when used in combination with the fragrance primer.

FIG. IV illustrates the increased substantivity of a commercially available woody, oriental, fresh, citrus and fruity fragrance when used in combination with the fragrance primer.

FIG. V illustrates the increased substantivity of a commercially available woody and crispy fragrance when used in combination with the fragrance primer.

FIG. VI illustrates the increased substantivity of a commercially available oriental and classical fragrance when used in combination with the fragrance primer.

FIG. VII illustrates the increased substantivity of a commercially available aromatic, fougere, fresh, and fruity fragrance when used in combination with the fragrance primer.

FIG. VIII illustrates the increased substantivity of a commercially available soft, oriental, and rich fragrance when used in combination with the fragrance primer.

FIG. IX illustrates the increased substantivity of a commercially available soft, oriental, and classical fragrance when used in combination with the fragrance primer.

FIG. X illustrates the increased substantivity of commercially available aromatic, fougere, and fresh fragrance when used in combination with the fragrance primer.

FIG. XI illustrates the increased substantivity of a commercially available water and crisp fragrance when used in combination with the fragrance primer.

FIG. XII illustrates the increased substantivity of a commercially available citrus and classical fragrance when used in combination with the fragrance primer.

### DETAILED DESCRIPTION OF THE INVENTION

According to the present invention, a fragrance is integrated into a suitable carrier such as silicone base or in a hydroalcoholic solution to provide a fragrance primer. The fragrance primer may contain any number of fragrance materials from all twelve fragrance families, which include fruity, floral, marine/ozonic, aldehydic, animalic leathery, musky, woody, sweet ambery, citrus, green, spicy and herbaceous.

The fragrance is not “built”, as known by one skilled in the art and described above, with a top middle and base structure, but the fragrance composition of the present invention is designed so the fragrance is rounded and not one ingredient is pronounced, which would give a specific odor or the family character.

When used in combination with certain commercially available fragrances, the fragrance primer of the present invention makes them longer lasting, more susbstantive and more diffusive. For other commercially available fragrances it pushes and reveals a facet forgotten by the habituated user of the fine fragrance. For other commercially available fragrances, the fragrance primer provides a new facet to that fragrance, such as more body for a fresh fragrance, more top note for a heavy fragrance, more modernity to an old-fashioned fragrance. This is supported by the analytical data presented in FIGS. I to XII, which illustrate that the fragrance primer of the present invention is long lasting and that it adds something to other fragrances.

Any number of the fragrances ingredients from the twelve fragrance families can be employed in the present invention, the only limitation being the compatibility with the carrier material.

Suitable fragrances include but are not limited to fruits such as almond, apple, cherry, grape, pear, pineapple, orange, strawberry, raspberry; musk, flower scents such as lavender-like, rose-like, iris-like, and carnation-like. Other pleasant scents include herbal scents such as rosemary, thyme, and sage; and woodland scents derived from pine, spruce and other forest smells. Fragrances may also be derived from various oils, such as essential oils or absolutes, or from plant materials such as peppermint and the like, from resins such as incense or galbanum resinoids or as described in “The perfume plants and essential oils in Grasse”, 1997, by Guy Gilly or from various molecules as described in “The Chemistry of Fragances” 1999 by David H. Pybus and Charles S. Sell, or in “Perfume and Flavor Chemicals” vol I and II, 1969, by Steffen Arctander.

A list of suitable fragrances is also provided in U.S. Pat. Nos. 4,534,891, 5,112,688 and 5,145,842, the contents of which are hereby incorporated by reference. Another source of suitable fragrances is found in Perfumes Cosmetics and Soaps, Second Edition, edited by W. A. Poucher, 1959. Among the fragrances provided in this treatise are acacia, cassie, chypre, cylamen, fern, gardenia, hawthorn, heliotrope, honeysuckle, hyacinth, jasmine, lilac, lily, magnolia, mimosa, narcissus, freshly-cut hay, orange blossom, orchids, reseda, sweet pea, trefle, tuberose, vanilla, violet, wallflower, and the like.

As used herein olfactory effective amount is understood to mean the amount of compound in perfume compositions the individual component will contribute to its particular olfactory characteristics, but the olfactory effect of the fragrance composition will be the sum of the effects of each of the fragrance ingredients. Thus the compounds of the invention can be used to alter the aroma characteristics of the perfume composition by modifying the olfactory reaction contributed by another ingredient in the composition. The amount will vary depending on many factors including other ingredients, their relative amounts and the effect that is desired.

According to one embodiment of the invention the fragrance primer may contain a fragrance material selected from one or all twelve of the fragrance families.

In a one embodiment the fragrance primer may contain the following amounts of fragrance ingredients, up to 50% of a fruity material, up to 90% of a floral material, up to 50% of a marine/aldehydic/ozonic material, up to 50% of an animalic leathery material, up to 90% of a musky material, up to 90% of a woody material, up to 90% of a sweet ambery material, up to 90% of a citrus material, up to 50% of a green material, up to 50% of a spicy material and up to 50% of a herbaceous material.

In a preferred embodiment the fragrance primer may contain the following amounts of fragrance ingredients, up to 5% of a fruity material, up to 25% of a floral material, up to 5% of a marine/aldehydic/ozonic material, up to 2% of an animalic leathery material, up to 60% of a musky material, up to 10% of a woody material, up to 5% of a sweet ambery material, up to 15% of a citrus material, up to 5% of a green material, up to 5% of a spicy material and up to 5% of a herbaceous material.

According to one embodiment of the invention the fragrance primer comprises from about 0.01% to about 15% and more preferably about 3% to about 9% of fragrance, with the remainder being a suitable carrier.

In additional embodiments, the fragrance primer can also be sprayed dried in combination with a starch material. Spray-drying is the most common encapsulation technique used to stabilize volatile substances such as flavors or fragrances, by encapsulating them in a solid form that is suited to many applications. Spray-dried powders are commonly made in the usual spray-drying equipment. Spray-drying is usually effected by means of a rotating disc or of multi-component nozzles. The fragrance primer may be dispersed in a polymeric carrier material. Non limiting examples of the latter include polyvinyl acetate, polyvinyl alcohol, dextrins, natural or modified starch, vegetable gums, pectins, xanthanes, alginates, carragenans or yet cellulose derivatives such as for example carboxymethyl cellulose, methylcellulose or hydroxyethylcellulose, and generally all materials currently used for encapsulation of volatile substances. The fragrance primer is then spray-dried in order to form a powder. This technique does not require a more detailed description herein, as it relies on conventional spray-drying techniques, which are perfectly well documented in the prior art, such as is described, e.g., in the Spray-Drying Handbook, 3rd ed., K. Masters; John Wiley (1979), and as is currently applied in the food industry or in the flavor and perfume industries.

In this embodiment the fragrance primer may contain from about 0.01% to about 15%, more preferably about 3% to about 8% neat fragrance and from about 0.01% to 8%, more preferably about 3% to about 6% spray dried fragrance, with the remainder being the silicone oil base.

According to one embodiment of the invention, any suitable carrier or vehicle may be employed.

The appropriate carrier materials include but are not limited to an aqueous or hydroalcoholic bases which includes alcohols such as ethanol, methanol, and the like; dipropylene glycol, dipropylene glycol ethers, diethyl phthalate and isopropyl myristate. The level of water in these systems is intentionally kept to a minimum, preferably below 5 weight percent of the fragrance composition, more preferably below 1 weight percent and most preferably less than 0.1 weight percent. Persons with skill in the art will be able to formulate fragrance compositions within the scope of the present invention that contain no intentionally added water.

In a preferred embodiment of the invention, the hydroalcoholic solution for use in this invention can be a mixture 0-90 parts alcohol, or 0-90 parts water.

According to another embodiment of the invention, the carrier may be a “silicone oil” which is defined as any liquid silicone known to be useful in personal care products. The silicone oil may be selected from but not limited to, cyclopentasiloxane, dimethicone, cyclomethicone, dimethiconol, ethyl perfluorobutyl ether, polydimethylsiloxane and mixtures thereof. This material is commonly used in the making of shampoos, because it is known to be especially good at depositing on hair. However, any other suitable silicone oil may also be used, and the skilled person can readily provide such an oil.

In a preferred embodiment of the invention, the silicone oils for use in this invention are linear polydimethylsiloxanes having a molecular weight of from about 5970 to about 116500 g/mol. It is also contemplated by this invention that mixtures of such polydimethylsiloxanes may also be used.

The linear polydimethylsiloxane is additionally characterized by a viscosity from about 100 cst to about 60000 cst, preferably from about 2000 cst to about 15000 cst. All viscosities mentioned herein are measured at 25° C. by means of a Brookfield RVT Viscometer. Spindles and speeds appropriate to the particular viscosity were used, as is the well-known practice in the art; for example, Spindle No. 5 and speed 20 were used for measurements in the preferred range between from about 6000 to about to about 12000 centipoise.

Another preferred silicone oil is a solution of polydimethylsiloxane gum in polydimethylsiloxane fluid, in an especially preferred version of this oil, the viscosity of the gum is greater than about 1,000,000 centistokes, the viscosity of the fluid is from about 10 to about 100,000 centistokes and the ratio of gum to fluid is from about 30:70 to about 70:30, preferably from about 40:60 to about 60:40.

A simple method to determine the solubility of fragrance raw materials in silicone is to determine it experimentally. However, in view of the vast number of perfumery materials available and since they can be mixed in various proportions to form a complete fragrance, it is desirable to have a theoretical method of determining the solubility of perfumery raw materials into silicone.

One such method is the Hansen solubility parameter (HSP) method, which is useful in predicting the solubility of perfumery materials. HSP may be evaluated as disclosed, for 5 example, in “Hansen Solubility Parameters: A User's Handbook” (Charles M. Hansen, CRC Press, 2000) or/and by using a software available on the market, such as Molecular Modeling Pro from www.Chemistry-Software.com or Hansen Solubility from Dynacomp Software.

### EXAMPLE Ia

### Composition of the Fragrance Primer Oil

A sample fragrance formulation was prepared to used in the fragrance primer system. The amounts are listed as percentages in weight.

| Fragrance Family | Weight Percent |
|---|---|
| Fruity | 1.52% |
| Floral | 20.67% |
| Marine/Aldehydic/Ozonic | 4.17% |
| Animalic Leathery | 0.14% |
| Musky | 51.04% |
| Woody | 5.06% |
| Sweet Ambery | 4.54% |
| Citrus | 10.25% |
| Green | 1.71% |
| Spicy | 0.07% |
| Herbaceous | 0.15% |
| Preservatives (BHT, Tinogard) | 0.68% |
| Total | 100% |

### EXAMPLE Ib

### Composition of the Fragrance Primer Oil

A sample fragrance formulation was prepared to used in the fragrance primer system. The amounts are listed as percentages in weight.

| Fragrance Family | Weight Percent |
|---|---|
| Fruity | 0.13% |
| Floral | 24.37% |
| Marine/Aldehydic/Ozonic | 1.51% |
| Animalic Leathery | 0.21% |
| Musky | 61.86% |
| Woody | 1.90% |
| Sweet Ambery | 0.77% |
| Citrus | 8.73% |
| Green | 0.26% |
| Spicy | 0.19% |
| Herbaceous | 0.07% |
| Total | 100% |

### EXAMPLE II

### Analytical Study of the Increased Substantivity of Fragrance when Used in Combination with Fragrance Primer

### Applied Dosage:

Fragrance Primer 50 μl at 10% in the silicone base
Fragrances: depends on the concentration in alcohol of the fragrance oil, for instance the commercially available soft, floral, rich and bouqouet fragrance was applied at a level of 100 μl.
Results illustrated in FIGS. I to XII are expressed in nanograms of materials emitted from the skin per second, between 0 and 30 minutes, 30 and 60 minutes and 1 hour and hour.

What is claimed:

1. A fragrance primer composition comprising fragrance material and carrier selected from the group consisting of a silicone base, a hydroalcoholic base and mixtures thereof wherein the fragrance material comprises up to 50% of a fruity material, up to 90% of a floral material, up to 50% of a marine/aldehydic/ozonic material, up to 50% of an animalic leathery material, up to 90% of a musky material, up to 90% of a woody material, up to 90% of a sweet ambery material, up to 90% of a citrus material, up to 50% of a green material, up to 50% of a spicy material and up to 50% of a herbaceous material.

2. The fragrance primer of claim 1 wherein fragrance material comprises of up to 5% of a fruity material, up to 25% of a floral material, up to 5% of a marine/aldehydic/ozonic material, up to 2% of an animalic leathery material, up to 60% of a musky material, up to 10% of a woody material, up to 5% of a sweet ambery material, up to 15% of a citrus material, up to 5% of a green material, up to 5% of a spicy material and up to 5% of a herbaceous material.

3. The fragrance primer of claim 1 wherein the silicone base is selected from the group consisting of cyclopentasiloxane, dimethicone, cyclomethicone, dimethiconol, ethyl perfluorobutyl ether,
polydimethylsiloxane and mixtures thereof.

4. The fragrance primer of claim 1 wherein the fragrance materials are spray dried.

5. The fragrance primer of claim 1 comprising from about 0.01% to about 15% of the fragrance material.

6. The fragrance primer of claim 4 wherein the fragrance primer comprises a neat fragrance material is from about 0.01% to about 15% and from about 0.01% to about 8% of a spray dried fragrance material.

7. A method of increasing the substantivity of a fragrance comprising the steps of
applying a fragrance primer to the skin;
applying a fragrance formulation over the fragrance primer.

8. The method of claim 7 wherein the fragrance primer comprises a fragrance formulation in a silicone base.

9. The method of claim 7 wherein the fragrance primer comprises a fragrance formulation in a hydroalcoholic base.

10. The method of claim 7 wherein the fragrance primer comprises a fragrance material and a silicone base wherein the fragrance material comprises up to 50% of a fruity material, up to 90% of a floral material, up to 50% of a marine/aldehydic/ozonic material, up to 50% of an animalic leathery material, up to 90% of a musky material, up to 90% of a woody material, up to 90% of a sweet ambery material, up to 90% of a citrus material, up to 50% of a green material, up to 50% of a spicy material and up to 50% of a herbaceous material.

11. The method of claim 9 wherein the fragrance primer comprises up to 5% of a fruity material, up to 25% of a floral material, up to 5% of a marine/aldehydic/ozonic material, up to 2% of an animalic leathery material, up to 60% of a musky material, up to 10% of a woody material, up to 5% of a sweet ambery material, up to 15% of a citrus material, up to 5% of a green material, up to 5% of a spicy material and up to 5% of a herbaceous material.

12. The method of claim 8 wherein the silicone base is selected from the group consisting of cyclopentasiloxane, dimethicone, cyclomethicone, dimethiconol, ethyl perfluorobutyl ether, polydimethylsiloxane and mixtures thereof.
